(19) United States
(12) Patent Application Publication
(10) Pub. No.: US 2019/0328237 A1
(43) Pub. Date: Oct. 31, 2019

(54) DEVICE AND METHOD FOR MONITORING SKIN TEMPERATURE AND CONDUCTANCE

(71) Applicant: Research Foundation of the City University of New York, New York, NY (US)

(72) Inventors: Chaya Edelman, Brooklyn, NY (US); Cira Cardaci, Staten Island, NY (US); Bo Guan, Bronx, NY (US); Mican Meneses, Toms River, NJ (US)

(21) Appl. No.: 16/397,708

(22) Filed: Apr. 29, 2019

Related U.S. Application Data
(60) Provisional application No. 62/663,697, filed on Apr. 27, 2018.

(51) Int. Cl. A61B 5/01; A61B 5/00

(52) U.S. Cl. CPC A61B 5/01; A61B 5/681; A61B 5/053; A61B 5/441; A61B 5/6831; A61B 5/6824

(57) ABSTRACT

A device for periodically recording skin temperature and conductance of a human subject. The device may be worn on the body (e.g. a pendant or wrist band) and temperature and conductance data is obtained and stored at predetermined intervals. Upon connection to a nearby smart device, the stored data is uploaded and subjected to additional processing. Various conditions of the human subject can be detected by processing the uploaded data.

### CROSS-REFERENCE TO RELATED APPLICATIONS

This application claims priority to and is a non-provisional of U.S. Patent Application 62/663,697 (filed Apr. 27, 2018) the entirety of which is incorporated herein by reference.

### BACKGROUND OF THE INVENTION

The subject matter disclosed herein relates to the monitoring to physiological conditions of human subjects and specifically to monitoring of temperature and electrical conductivity. People experience a wide variety of skin conditions that should be monitored. For example, degree of skin hydration, concentration of skin oils and increases in skin temperature (e.g. fever) have all been linked to undesirable physiological states. Prior attempts have been made to monitoring these skin conditions but, to date, no solution has been found to be entirely satisfactory. Additional solutions are therefore desirable.

The discussion above is merely provided for general background information and is not intended to be used as an aid in determining the scope of the claimed subject matter.

### SUMMARY

A device for periodically recording skin temperature and conductivity of a human subject is provided. The device may be worn on the body (e.g. a pendant or wrist band) and temperature and conductivity data is obtained and stored at predetermined intervals. Upon connection to a nearby smart device, the stored data is uploaded and subjected to additional processing. Various conditions of the human subject can be detected by processing the uploaded data.

In a first embodiment, a method for detecting a hot flash is provided. The method comprising: placing a device proximate a human, the device comprising: a temperature sensor; a conductivity sensor; a power source; a wireless transmitter; a data storage device; a processor. The method further comprises monitoring a temperature of the human over at a predetermined time of at least one minute and less than five minutes using the temperature sensor; determining a rate of temperature change of the human over the predetermined time; monitoring a skin conductance of the human over the predetermined time using the conductivity sensor; determining a rate of skin conductance change of the human over the predetermined interval; detecting a presence of a hot flash if both (1) the rate of temperature change over the predetermined time is greater than 1° C. per minute and (2) the rate of skin conductance change over the predetermined time is greater than 3 microSiemens per minute; or detecting an absence of a hot flash if either (1) the rate of temperature change over the predetermined time is less than or equal to 1° C. per minute or (2) the rate of skin conductance change over the predetermined time is less than or equal to 3 microSiemens per minute.

In a second embodiment, a device for detecting a physiological condition of a human is provided. the device comprising: a conductivity sensor for continually sensing conductivity readings over a predetermined time; a temperature sensor for continually sensing temperature readings over the predetermined time; a power source; a wireless transmitter for wirelessly transmitting the conductivity readings and the temperature readings; a data storage device; a processor configured to continually store the conductivity readings and the temperature readings on the data storage device, thereby producing stored data the processor further configured to wirelessly transmit the stored data using the wireless transmitter.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a top view of a device for sensing skin conductivity and temperature;

FIG. 2 is a bottom view of the device;

FIG. 3 is a perspective view of the device with the lid attached;

FIG. 4 is a perspective view of the device with the lid removed;

FIG. 5 is a perspective view of another embodiment of the device;

FIG. 6 is a schematic depicting data flow through the system; and

FIG. 7 is schematic of the circuitry of the electronic platform that controls the device.

### DETAILED DESCRIPTION OF THE INVENTION

This disclosure pertains to a device that comprises a corneometry sensor that senses capacitance difference to obtain a conductivity measure and a thermistor that measures skin temperature. The disclosed device addresses a need to continuously monitor multiple parameters of skin condition in menopausal women, while educating and empowering the community in the process. In another embodiment the device is used for fever detection caused infection. In one such embodiment neutropenia fever in cancer patients is detected for early warning of infection due to neutropenia.

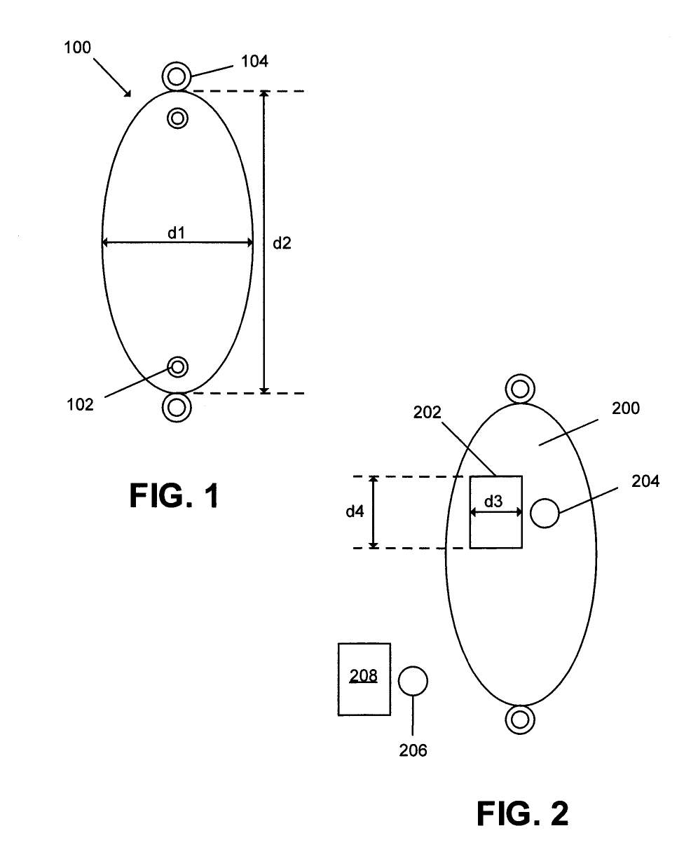

FIG. 1 is a top view of one embodiment of the device 100. The device 100 may have, for example, a width (d1) of about 27 mm and a height (d2) of about 60 mm. The device 100 comprises a bottom section and a lid that are removably secured to one another by fasteners 102 which may be snap connectors, screws or other means for fastening. The device 100 further comprises at least one loop 104 for receiving a chain or string such that the device 100 can be worn as a pendent or bracelet. FIG. 2 is a bottom view of the device 100. In FIG. 2, the device is shown with a bottom surface 200 that has a first opening 202 and a second opening 204. An electrical conductivity sensor 208 fits within the first opening 202. The first opening may be square with a width (d3) of about 11 mm and a height (d4) of about 15 mm.

A temperature sensor 206 fits into the second opening 204. In one embodiment the temperature sensor 206 is an infrared (IR) temperature sensor. The second opening 204 may have, for example, a diameter of about 6 mm. In one embodiment, the user presses the device 100 against the user's skin and conductivity data and temperature data are obtained using the respective sensor. In another embodiment the device 100 continuously acquires data. As used in this specification, the term “continuous” refers to the repeated acquisition of data points at a predetermined interval (e.g. once per second, at least once per five seconds, etc.) throughout a predetermined time period (e.g. 1-5 minutes) The data is first stored first locally within memory of device 100 and subsequently transmitted to the internet cloud or nearby smart device. The transmission may be wired transmission or wireless transmission.

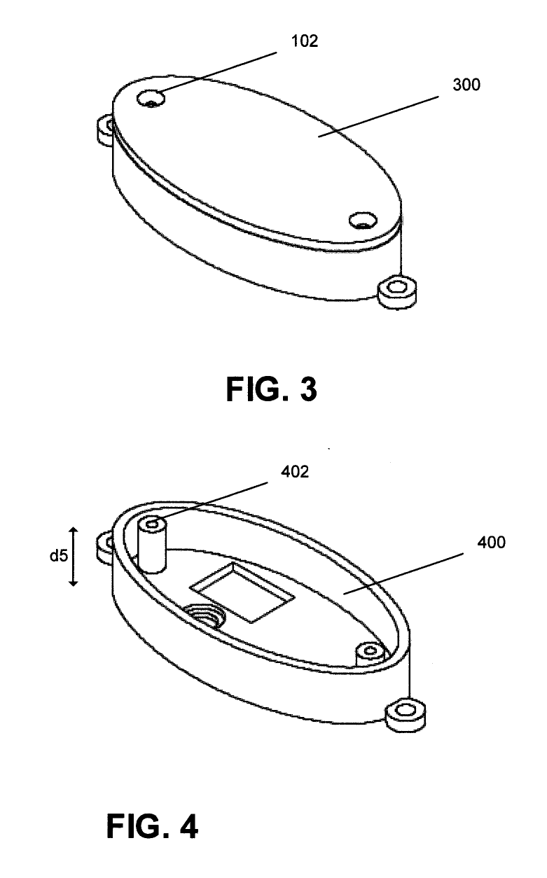

FIG. 3 is a perspective view of the device 100 with the lid 300 in place. FIG. 4 is the same perspective view except in that lid 300 has been removed to better illustrate the bottom section 400. The internal components (e.g. the temperature sensor 206, conductivity sensor 208 and electronic platform 606) have been omitted for clarity of illustration. Fasteners 102 (which are holes in the embodiment of FIG. 3) allow a screw to pass through the lid 300 and engage with threads 402. In one embodiment, the bottom section 400 has an outer depth (d5) of about 11 mm. The lid 300 may add, for example, an additional 2 mm for a total outer depth of about 13 mm.

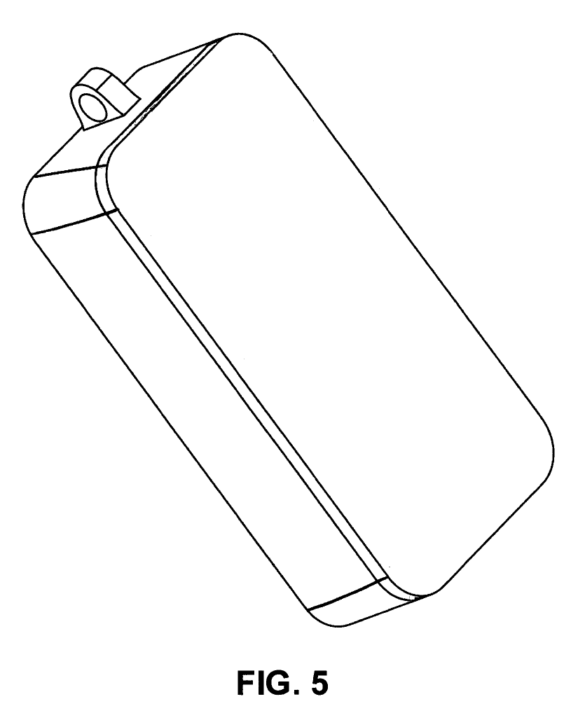

FIG. 5 depicts another embodiment of the device wherein the bottom section and the lid have a different shape. Alternative shapes would be apparent to those skilled in the art after benefitting from reading this specification.

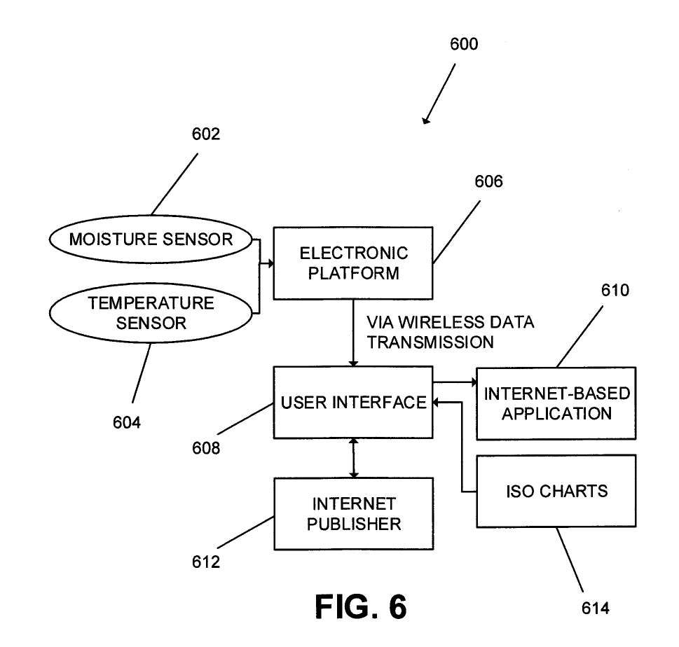

FIG. 6 depicts a system 600 that comprises a conductivity sensor 602 and a temperature sensor 604 that provides conductivity data and temperature data to an electronic platform 606. In one embodiment, the electronic platform 606 is an ARDUINO® brand electronic platform and includes a single board microcontroller, a power supply (e.g. a rechargeable or replaceable battery such as a coin cell battery) and a wireless module for wirelessly exchanging data. The wireless module may use radio waves (e.g. Wi-Fi) or infrared waves (e.g. BLUETOOTH®). The electronic platform 606 is disposed inside the device 100. In one embodiment, the device 100 uses iOS for data processing and software interface. The device 100 is wirelessly connected to a user interface application 608 (such as an iOS application stored on a nearby smart device (e.g. a smartphone, tablet or desktop computer)) which displays the collected skin data and provides education materials to the user. The temperature and conductivity data may be used to calculate the rate of change of the temperature and the conductivity. In one embodiment, these calculations are performed using a processor within the electronic platform 606. In another embodiment, these calculations are performed using a processor within the nearby smart device. The user interface application 608 transmits data to an internet-based application 610 which may be, for example, a FIREBASE® brand internet-based application. Data from the user interface application 608 can be published online using internet publisher 612 (e.g. WORDPRESS® internet publisher). The data exchange between the user interface application 608 and the internet publisher 612 is a two-way data exchange. ISO charts 614 (e.g. ISO charts by Daniel Cohen) is a framework that converts uploaded data from FIREBASE into a user-interface-friendly line and bar graphs. Such ISO chart frameworks are available under the APACHE® license.

The device 100 obtains temperature data and conductivity data at predetermined intervals (e.g. once per minute, 3 times per hour, etc.) and temporarily stores the temperature data within the electronic platform 606. During each measurement, the temperature and conductivity is monitored over a predetermined time of at least one minute and less than five minutes. When the electronic platform 606 wirelessly connects to the user interface application 608, the accumulated temperature data is transmitted to the user interface application 608. In a similar fashion, conductivity data is also obtained, temporarily stored and then wirelessly transmitted. The user interface 608 is configured to process the temperature data and the conductivity data. The temperature data and/or conductivity data can be presented to the user as a function of time (e.g. as a numeric table or as a graph) showing how temperature and conductivity have change over time.

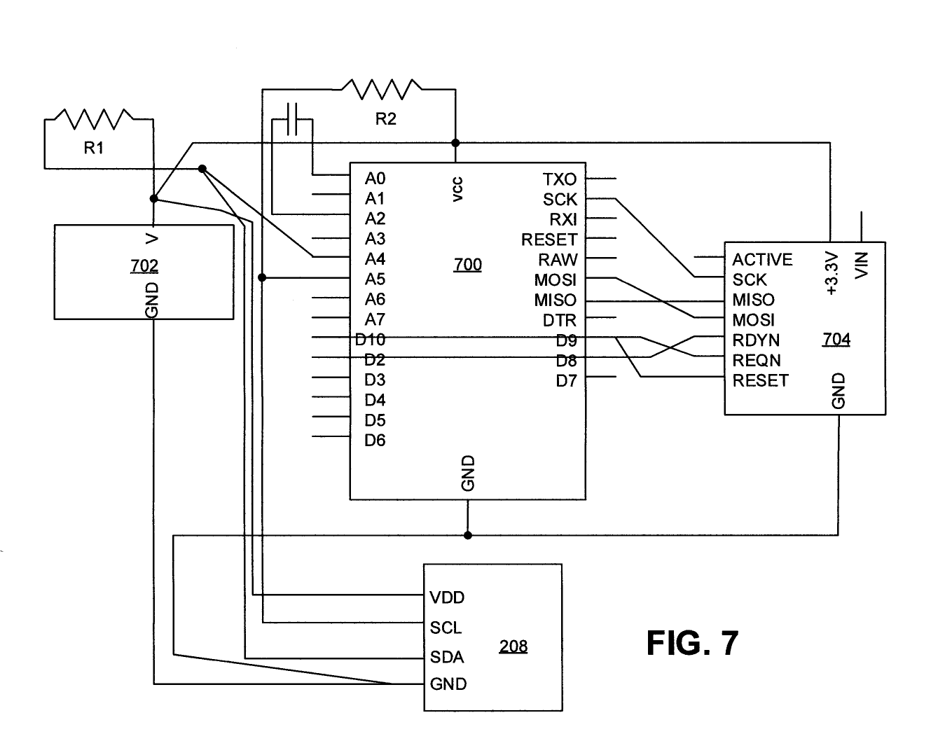

The schematic of FIG. 7 shows the microcontroller 700 that stores a program to run the temperature sensor 206 and conductivity sensor 208. The schematic also shows an interdigital capacitor to receive temperature data and conductivity data. The electrical components are powered by the power source 702. A wireless module 704 communicates with the user interface application 608. The two resistors that are depicted in FIG. 7 may be 10 kΩ resistors. Programming of the microcontroller 700 is done through a programming software such as ARDUINO® IDE.

The device finds applications in a variety of fields including (1) monitoring skin conductivity and temperature for acne patients; (2) monitoring skin condition of all populations; (3) monitoring skin condition in harsh climates; (4) skin condition tracking for postmenopausal and premenopausal women; (5) collecting and selling skin condition data; (6) skin monitoring of circulation or endocrine issues; (7) testing of skin treatment/medication efficacy; (8) monitoring clinical trial use; (9) monitoring of dermatological conditions (i.e. acne, rosacea, cellulitis, anaphylaxis etc.); (10) monitoring of medical conditions (cardiovascular disease, hypothyroidism, panic disorder, drug/alcohol dependence etc.); (11) monitoring of skin parameters and peripheral parameters of the body (i.e. temperature) during emergency or trauma situations; and (12) as a supplement to virtual reality (VR) and/or augmented reality applications (games, educational training, etc.).

Surface temperature of skin can increase up to 5-7 degrees Celsius during a hot flash. This increase in temperature can be found in a variety of body areas including the face, neck, chest, as well as tip of the fingers. The increase in temperature can last usually for 1-5 minutes but can be up to half an hour. Subjective reporting of hot flash is not always accurate and does not correlate well with objective measures. Another indicator for hot flash is skin moisture as perspiration is a common physiological sign typically measured by skin electrical conductance. Research has shown that 98% of 128 subjective flashes in 8 postmenopausal women were accompanied by elevations in sternal skin conductance compared to only 82% for finger temperature. The disclosed device takes into account both skin temperature change and skin conductance in tracking a hot flash. A peripheral skin temperature that exceeds 1 degree Celsius within 5 minutes (minimum rate of 0.2 degrees C. per min) from the baseline, coupled with a moisture measurement of skin of a conductance increases of more than 5 microSiemens from the baseline also within three minutes (minimum rate of 3 microSiemen per min), indicate hot flash. A hot flash, in turn, is the most common symptom of the menopausal transition period.

In one embodiment, the device continuously monitors the user for changes in temperature and conductivity. In another embodiment, the device monitors the user for a predetermined period of time after actuation of the device. For example, the device may be pressed tightly against the user's skin and the skin's conductivity is detected. This triggers the beginning of a five-minute period during which the device will continually monitor for changes in temperature and conductivity. Based on changes in these parameters, a hot flash can either be detected or refuted.

The smart device that receives the data can log and present the changes in the data over prolonged periods of time (e.g. a week, a month, a year, etc.). The data may be presented in a tabular form (e.g. tables of numeric data) or a graphic form (e.g. graphs). This long term data can be presented to medical professionals and is useful in medical diagonistics.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for detecting a hot flash, the method comprising:
placing a device proximate a human, the device comprising:
a temperature sensor;
a conductivity sensor;
a power source;
a wireless transmitter;
a data storage device;
a processor;
monitoring a temperature of the human over at a predetermined time of at least one minute and less than five minutes using the temperature sensor;
determining a rate of temperature change of the human over the predetermined time;
monitoring a skin conductance of the human over the predetermined time using the conductivity sensor;
determining a rate of skin conductance change of the human over the predetermined interval;
detecting a presence of a hot flash if both (1) the rate of temperature change over the predetermined time is greater than 1° C. per minute and (2) the rate of skin conductance change over the predetermined time is greater than 3 microSiemens per minute; or
detecting an absence of a hot flash if either (1) the rate of temperature change over the predetermined time is less than or equal to 1° C. per minute or (2) the rate of skin conductance change over the predetermined time is less than or equal to 3 microSiemens per minute.

2. The method as recited in claim 1, further comprising wirelessly transmitting the temperature and the skin conductance to a smart device.

3. The method as recited in claim 2, wherein the smart device performs the step of determining the rate of temperature change and the step of determining the rate of skin conductance change.

4. The method as recited in claim 3, wherein the smart device performs the step of detecting a presence of the hot flash.

5. The method as recited in claim 4, wherein the smart device performs the step of detecting an absence of the hot flash.

6. The method as recited in claim 1, wherein the step of monitoring the temperature occurs continually throughout the predetermined time such that at least one temperature reading is obtained every five seconds.

7. The method as recited in claim 1, wherein the step of monitoring the skin conductance occurs continually throughout the predetermined time such that at least one skin conductance reading is obtained every five seconds.

8. The method as recited in claim 1, wherein the step of monitoring the temperature occurs continually throughout the predetermined time such that at least one temperature reading is obtained every second.

9. The method as recited in claim 1, wherein the step of monitoring the skin conductance occurs continually throughout the predetermined time such that at least one skin conductance reading is obtained every second.

10. A device for detecting a physiological condition of a human, the device comprising:
a conductivity sensor for continually sensing conductivity readings over a predetermined time;
a temperature sensor for continually sensing temperature readings over the predetermined time;
a power source;
a wireless transmitter for wirelessly transmitting the conductivity readings and the temperature readings;
a data storage device;
a processor configured to continually store the conductivity readings and the temperature readings on the data storage device, thereby producing stored data the processor further configured to wirelessly transmit the stored data using the wireless transmitter.

11. The device as recited in claim 10, wherein the device is a pendant or a wrist band.

12. The device as recited in claim 11, wherein the temperature sensor is an infrared temperature sensor that is at least partially exposed through a surface of the device.

13. The device as recited in claim 12, wherein the conductivity sensor is at least partially exposed through a surface of the device.

14. A system for detecting a hot flash, the system comprising;
the device as recited in claim 10;
a smart device for wirelessly receiving the temperature readings and the conductivity readings.

15. The system as recited in claim 14, wherein the device is a pendant or a wrist band.

16. The system as recited in claim 14, wherein the smart device is a smart phone.
